(19) United States
(12) Patent Application Publication
(10) Pub. No.: US 2012/0064097 A1
(43) Pub. Date: Mar. 15, 2012

(54) Enhanced Binding of Pro-Inflammatory Cytokines by Polysaccharide-Antibody Conjugates

(76) Inventors: Newell R. Washburn, Pittsburgh, PA (US); Liang Tso Sun, Pittsburgh, PA (US)

(21) Appl. No.: 13/186,925

(22) Filed: Jul. 20, 2011

Related U.S. Application Data
(60) Provisional application No. 61/365,941, filed on Jul. 20, 2010.

(51) Int. Cl. A61K 39/44; C07K 17/08; A61P 29/00; C07K 17/10

(52) U.S. Cl. 424/178.1; 530/391.1; 525/54.1

(57) ABSTRACT

We provide monoclonal antibodies against interleukin-1β and tumor necrosis factor-α that remain biologically active in vitro when conjugated to high molecular weight polysaccharides. We report enhanced binding of these cytokines when their monoclonal antibodies are conjugated to alginate compared to non-conjugated monoclonal antibodies. In cell assays, polysaccharide-antibody constructs of the invention inhibited cytokine signaling to comparable levels as that of unmodified antibodies. Conjugation of cytokine-neutralizing antibodies to high molecular weight polymers enhances the affinities cytokine-binding moieties used as anti-inflammatory therapeutics.

### CROSS-REFERENCE TO RELATED APPLICATIONS

This application claims priority to U.S. Provisional Patent Application No. 61/365,941, filed on Jul. 20, 2010, and incorporated by reference herein.

### STATEMENT REGARDING FEDERALLY SPONSORED RESEARCH OR DEVELOPMENT

This invention was made using funds provided by National Institutes of Health grant no. NIH R43GM085897 (NRW). The United States government may have some rights in this invention.

### BACKGROUND OF THE INVENTION

1. Field of the Invention

Compositions and methods for treating degenerative inflammatory conditions by enhanced inhibition of inflammatory mediators are disclosed.

2. Background of the Related Art

Biological tissue is comprised of cells and extracellular matrix. The structure and strength of tissue is a consequence of the interaction between the cells and the extracellular matrix. The extracellular matrix is comprised of proteins and glycoproteins such as collagen, elastin, fibronectin, vitronectin and laminin; polysaccharides such as the glycosaminoglycan (“GAG”) hyaluronic acid; and proteoglycans such as aggrecan, decorin and perlecan. Cells attach directly to each other and to the extracellular matrix, which supports the attachment of cells, serves as a scaffold or structural support for cells, mechanically regulates cellular functions via cell adhesion, lubricates cells and provides a transport system for intercellular mediators, nutrients and waste products.

Acute wounds normally heal in an orderly and efficient manner by progressing through four distinct but overlapping phases: hemostasis, inflammation, proliferation and remodeling. Throughout these phases, cells and the extracellular matrix (“ECM”) play an important role in regulating and integrating many key processes of healing.

The hemostasis phase of wound healing involves the formation of a provisional wound matrix. The clot that forms at the site of an injury not only stops bleeding, but deposits a host of plasma and cell-secreted constituents at the wound interface. Epidermal cells subsequently dissect their way under the clot and over the granulation tissue (which is comprised of a dense population of macrophages, fibroblasts and newly formed blood vessels embedded in a loose matrix of fibrin, fibronectin, collagen and other ECM proteins). Stimulation of the clotting cascade results in the proteolytic cleavage of fibrinogen by the enzyme thrombin, forming an insoluble fibrin clot that holds damaged tissues together and provides the provisional matrix. In addition, the clot contains fibronectin molecules that are present in plasma and bind to fibrin through fibrin-specific binding sites.

The inflammatory response involves the migration of neutrophils, macrophages and lymphocytes to the site of the injury. Neutrophils are the first inflammatory cells to respond to the soluble mediators released by platelets and the coagulation cascade. Their primary role is to mount the first line of defense against infection by phagocytosing and killing bacteria, and by breaking down foreign materials and devitalized tissue. Neutrophils also produce and release inflammatory mediators such as tumor necrosis factor alpha (“TNF-α”) and interleukin-1 (“IL-1”), which further recruit and activate neutrophils and macrophages. In this way early inflammatory signals can induce massive responses at the site of injury through positive signaling loops that involve these soluble signaling proteins. Neutrophils also produce and contain high levels of proteases and oxygen free radicals, which they use to break down the surrounding tissue. In healthy patients, this process is necessary to establish the proper environment for the later stages of tissue repair. Neutrophils release these substances into the local wound area upon cell death, which can cause extensive tissue damage and prolong the inflammatory phase. The persistent presence of high levels of bacteria in a wound may contribute to chronicity through continued recruitment of neutrophils and their release of proteases, cytokines and intracellular contents.

Neutrophils are usually depleted in an acute wound after two to three days and are replaced by tissue macrophages. Tissue macrophages function as phagocytes that ingest bacteria, devitalized tissue and depleted neutrophils, and produce collagenases and elastase to enzymatically mediate the degeneration of devitalized tissues. They are able to regulate proteolytic destruction of tissue in the wound by producing and secreting inhibitors for these enzymes.

Fibroblasts migrate into the provisional wound matrix as part of angiogenesis and in response to chemotactic growth factors released by platelets from the wound area and subcutaneous tissue and begin to express new integrin receptors. The integrin receptors then generate new intracellular signals that stop the fibroblasts from migrating. Growth factors and proteins contained within the provisional wound matrix help to stimulate fibroblasts to begin proliferating and synthesizing new collagen and other ECM components. In this way the provisional wound matrix functions as a reservoir to help trap growth factors and actively signals fibroblasts, epidermal cells and vascular endothelial cells, via their integrin receptors, to transform into activated wound cells that will repair the injury.

Macrophages also mediate the transition from the inflammatory phase to the proliferative phase of normal healing. They release a wide variety of growth factors and cytokines, including TNF-α, transforming growth factor beta (“TGF-β”), platelet-derived growth factors (“PDGFs”), IL-1, interleukin six (“IL-6”), insulin-like growth factor-one (“IGF-1”) and fibroblast growth factor (“FGF”). Some of these soluble mediators recruit and activate fibroblasts, which will synthesize, deposit and organize the new tissue matrix, while others promote angiogenesis. FIG. 1 illustrates the M1 and M2 macrophage phenotypes. The M1 phenotype produces pro-inflammatory mediators. The M2 phenotype produces pro-angiogenic factors and mediators of tissue repair and remodeling.

During the proliferative phase, the provisional wound matrix is remodeled and replaced with scar tissue, consisting of new collagen fibers, proteoglycans and elastin fibers, which partially restore the structure and function of the tissue. This is accomplished by the migration, proliferation and differentiation of epithelial cells, fibroblasts and vascular endothelial cells from adjacent uninjured tissue and stem cells that originate in the bone marrow and circulate to the wound site.

Fibroblasts migrate into the wound in response to soluble cytokines and growth factors, which are initially released from platelets when they degranulate and later by macrophages in the wound. These include PDGF, TGF-β and FGF. Fibroblasts secrete proteases called matrix metalloproteinases (“MMPs”) which are essential for the migration of cells through the ECM. For example, collagenase (“MMP-1”) cuts intact collagen at a single site, gelatinases (“MMP-2” and “MMP-9”) degrade partially denatured collagen (gelatin), and stromelysin (“MMP-3”) degrades multiple protein substrates in the ECM. In addition, MMPs remove collagen and other ECM components that were denatured during the injury. Partially degraded collagen molecules will not bind properly with new collagen molecules synthesized during scar formation, resulting in disorganized, weak ECM, so the degraded collagen molecules must be removed by controlled action of the MMPs. However, this process must be carefully controlled by tissue inhibitors of metalloproteinases (TIMPs) enzymes, which prevent the MMPs from degrading intact, functional matrix.

After the fibroblasts have migrated into the provisional wound matrix, they proliferate and begin to synthesize new collagen, elastin, proteoglycans and other components that comprise granulation tissue. PDGF and TGF-β are two of the important growth factors that regulate the expression of ECM genes and proteases in fibroblasts. Cells from surrounding tissues begin to proliferate and migrate into the wound site, bind to the newly deposited matrix and form scar tissue.

Remodeling is the final phase of wound healing and occurs through the actions of several different classes of proteolytic enzymes such as MMPs and serine proteases produced by cells in the wound bed at different times during the healing process. Specific MMP proteases that are necessary for wound healing are the collagenases, which degrade intact fibrillar collagen molecules; the gelatinases, which degrade damaged fibrillar collagen molecules; and the stromelysins, which degrade proteoglycans. Under normal conditions, the destructive actions of proteolytic enzymes are carefully regulated by TIMP enzymes, which are produced by cells in the wound bed.

A chronic wound is a wound that does not heal in a normal manner and in a predictable amount of time. Several common medical conditions are associated with chronic wounds, including diabetic ulcers, decubitus/pressure ulcers, venous ulcers, severe burns, ischemia and anemia, for example. Statistics on patients diagnosed with diabetes, for example, indicate that more than 14.6 million diabetics are at an increased risk of foot ulceration at a rate of 15% at some point in their lifetime. This complication represents a significant fraction of the cost of caring for patients with diabetes, estimated to range from 25% to 50%, and diabetic foot ulcers are the most important risk factor for lower extremity amputation at a rate of approximately 15.6%.

Chronic wounds generally involve uncharacteristically slow healing, abnormal healing, or a complete lack of healing. Acute normal-healing wounds are characterized by a precise balance between degradation and regeneration of damaged tissue throughout the four phases of healing described above. Chronic wounds, however, are characterized by a retarded healing trajectory where the proliferative phase fails to initiate and a prolonged or indefinite inflammatory phase occurs. In the sustained inflammatory phase, degradation predominates and regeneration of the damaged tissue is minimal to non-existent. This lengthened inflammatory phase may cause increased levels of proteases such as MMPs, elastase, plasmin and thrombin. The increased levels of protein can destroy components of the ECM and damage the growth factors and their receptors that are essential for transitioning from the inflammatory phase to the proliferative phase of the healing response. Accordingly, chronic wounds are often characterized by incomplete closure and increased incidence of infection.

The pathophysiology of chronic wounds varies according to the underlying medical condition. However, all chronic wounds are characterized by several common features including increased levels of pro-inflammatory mediators relative to acute or properly healing wounds. Pro-inflammatory mediators are substances secreted by cells as part of a host response to disease or infection that promote inflammation. Pro-inflammatory mediators include prostaglandins, histamines, bradykinin, complement proteins, chemokines and cytokines such as IL-1, IL-6, IL-8, IL-12, IL-18, nitric oxide (“NO”), monocyte chemoattractant protein-1 (“MCP-1”), and interferon gamma (“INF-γ”), for example.

Increased levels of the cytokines IL-1β, IL-6 and TNF-α have been measured in chronic wounds relative to acute wounds. For example, in wound fluid from healing versus non-healing leg ulcers, the median concentration of IL-1β was found to be 7785 pg/ml versus 17,902 pg/ml, the median concentration of IL-6 was found to be 55,185 pg/ml versus 77,762 pg/ml, and the median concentration of TNF-α was found to be 1639 pg/ml versus 4734 pg/ml. In a comparison of chronic wounds and acute wounds from mastectomies, 100-fold increases in IL-1β and TNF-α and a six-fold increase in IL-6 were measured.

In acute wounds, IL-1β activates neutrophils, promotes chemotaxis, and stimulates cytokine production in the surrounding tissue. It has been demonstrated that by increasing the activity of IL-1β in knock-out (KO) mice lacking the IL-1β receptor antagonist (IL-1ra) increases neutrophil invasion at the wound site and significantly lengthens the time for wound closure. Comparisons of histology data in wild type (WT) and IL-1ra knock-out (KO) mice demonstrated that neutrophil recruitment in KO mice two days after wounding was nearly double that in WT mice. The data following wound closure over 14 days showed a significant inhibition of closure in the KO mice.

It has been demonstrated that wounds in IL-6 KO mice took up to three times longer to heal than those of WT controls. Wounds in these animals were characterized by a significant delay in re-epithelialization and inhibition of the formation of granulation tissue. A comparison of the area of the healing wound in WT versus IL-6 KO mice quantified over 15 days show that IL-6 KO mice had a five-day delay in the onset of healing.

It has also been reported that KO mice lacking the TNF-α receptor p55 showed accelerated wound healing, characterized by increases in re-epithelialization, collagen production, angiogenesis, and expression of TGF-β1, vascular endothelial growth factor, and connective tissue growth factor. Comparisons of wound closure in WT and TNF-α KO mice show that wound closure over a 14 day period was enhanced in the TNF-α KO mice. In WT mice, there was significant invasion of neutrophils up to 6 days, but in the KO mice, both neutrophil and macrophage invasion was significantly inhibited.

Strategies for treating chronic wounds include debridement, in which surface debris and necrotic tissue is removed, and topical application of growth factors, such as PDGF, FGF-2, and TGF-β. However, the underlying tissue often does not heal better than the debrided tissue, and topical application of free growth factors does not appear to be a substantially effective therapy.

Inhibiting pro-inflammatory cytokines has been shown to be an effective strategy for promoting healing of chronic wounds. Infliximab, a human monoclonal antibody that binds TNF-α and inhibits its signaling, was shown to promote wound closure in patients suffering from chronic ulcers, with 5 of 14 ulcers showing complete closure after 8 weeks. An antagonist against the IL-1β receptor, anakinra, has also been developed to treat inflammatory conditions, such as rheumatoid arthritis. Although the clinical trials showed some efficacy, anakinra has been found to be less effective in treating these conditions than therapeutics that target TNF-α. An important caveat is the contraindication against taking therapeutics that inhibit signaling from both TNF-α and IL-1β. When patients suffering from rheumatoid arthritis were given both etanercept and anakinra, there was a statistically insignificant (P=0.914) improvement in outcome compared to etanercept alone, but a significant increase in the risk of infection (0% for etanercept alone; 3.7-7.4% for the combination therapy) was observed.

Additional strategies for healing chronic wounds include using a viscous matrix consisting of hyaluronic acid covalently modified with the arginine-glycine-aspartic acid (“RGD”) peptide. Further strategies involve compositions consisting of biodegradable polymer, RGD peptide, and a ligand such as a ligand to a PDGF receptor, a ligand to an insulin receptor, a ligand to an interleukin four (“IL-4”) receptor, and a ligand to an IGF receptor.

Hyaluronic acid is a hydrophilic material that is recognized by the CD44 receptor and receptor for HA-mediated motility (RHAMM) in multiple cell types and promotes a motile phenotype. Furthermore, HA degradation products are known damage-associated molecular patterns that may recruit additional repair cells to the wound site. In clinical trials on diabetic foot ulcers, the HA-RGD matrix promoted complete healing in 35% (14 of 40) patients as compared to 8% (2 of 25) in the control group that received a saline solution placebo. Similar results were observed when RGD-HA was used to treat leg ulcers in patients with sickle-cell anemia. While these various therapies show improvements in patient outcomes, there remains a critical, unmet need for effective, and cost-effective, therapies to heal chronic wounds. Other conditions characterized by intense inflammation include burns, psoriasis, and Crohn's disease. While each of these conditions has different physiological roots, inflammatory responses and their mediators play central roles in all of them.

Conjugating polymers to therapeutic proteins has been reported as a way to modulate their pharmacokinetics in vivo and enhance their efficacy. The synthetic polymer poly(ethylene glycol) (PEG) has been primarily used to engineer polymer-protein therapeutics because of its biologically inert nature and excellent solubility in aqueous media. The primary objective of this type of conjugation is to prolong the circulation time of these molecules by reducing their renal clearance rates. The PEG molecular weights tend to range between 20-60 kDa, which appears to balance improvements in circulation time with retention of biological activities. These conjugated polymers could also protect therapeutic proteins from immune responses and enzymatic attack, thus increasing the stability of these therapeutic agents in vivo, with the overall goal of increasing the systemic activity of the proteins.

### BRIEF SUMMARY OF THE INVENTION

While PEGylation of therapeutic proteins is known at best to leave the binding affinity unchanged, in many cases a decrease in binding affinity has been reported. This is generally attributed to steric hindrance of the PEG chain blocking intermolecular binding interactions. We demonstrate here that covalent conjugation of cytokine-neutralizing antibodies to sodium alginate can significantly increase binding affinity for the cytokine. Useful cytokine-neutralizing molecules may include, for example, but are not limited to antibodies, antibody fragments, phage peptides, receptor fragments, or nucleic acid-based aptamers against tumor necrosis factor-a, interleukin-1a, interleukin-1b, interferon-a, interferon-g, interleukin-12, interleukin-23, transforming growth factor-b, interleukin-6, interleukin-2, and interleukin-4. Chemically modified alginates, including those with alkyl or aryl substituents via chemical linkages such as esters or amides, and propylene glycol-functionalized alginates, are also included. In preferred embodiments alkyl substituents are C1-C10 alkyl, preferably C1-C6 alkyl. Aryl substituents may be benzyl or benzyl-substituted substituents. Cross-linked versions, either through native binding of divalent ions (e.g. calcium) or through polymerizable groups, such as vinyl or allyl functionality, may also be used. It should be noted that cytokine-neutralizing molecules are a subset of cytokine-inhibiting molecules. It should also be noted that when the term “derivatives” is used it should be given its art-accepted meaning for the compound from which the derivative is derived, and may include but not be limited to derivatives including C1-C10 alkyl, preferably C1-C6 alkyl, as well as aryl and alkyl-substituted aryl, having alkyl substitution of C1-C10, preferably C1-C6.

### BRIEF DESCRIPTION OF THE FIGURES

FIG. 1 shows the monomer structures of hyaluronic acid, carboxymethylcellulose, and alginic acid (referred to herein as alginate).

FIG. 2 shows synthesis of polysaccharide-antibody conjugates.

FIG. 3 shows coupling of polysaccharides to monoclonal anti-cytokine antibodies. FIG. 3(A) shows non-denaturing polyacrylamide gels, stained with Alcian Blue, showing the results from analyzing hyaluronic acid (HA) samples with varying concentration with and without conjugated antibody. (i) 0.05% HA, (ii) 0.025% HA, (iii) 0.013% HA, (iv) 0.006% HA, (v) 0.003% HA, (vi) 0.1×HA-anti-IL1β mAb, (vii) 0.1×HA-anti-TNF-α mAb, (viii) 0.1×HA+mAb. FIG. 3(B) shows CMC concentration calibration curve fit to the equation y=0.0398x−0.0388, R²=0.9965. FIG. 3(C) shows HA concentration calibration curve fit to the equation y=0.0346x−0.0062, R²=0.9964.

FIG. 4 shows a fluorescence immunosorbent assay calibration curve used to measure the monoclonal antibody concentration in the polysaccharide-mAb samples. Data were fit to y=1.1572x+0.771 (R²=0.9878).

FIG. 5 shows PAGE analysis of alginate concentration in alginate-mAb solutions. Top: Image of gels that were analyzed to establish the calibration curve. Bottom: Calibration curve used to calculate concentration of alginate.

FIG. 6 shows a calibration curve used to determine antibody concentration in alginate-mAb conjugates.

FIG. 7 shows binding affinity analysis using Fortebio Octet system. (Top) Association, dissociation curve, and best-fit isotherms of different conditions: (A) anti-IL-1β mAb, (B) HA-anti-IL-1β mAb conjugate, (C) CMC-anti-IL-1β mAb conjugate. (Bottom) Plot of the residuals from the best-fit curve.

FIG. 8 shows binding affinity measurements of alginate-mAb constructs. FIG. 8(A) shows alginate-(anti-IL-1β) binding of IL-1β. FIG. 8(B) shows alginate-(anti-TNF-α) binding of TNF-α. The nearly flat dissociation curve (starting at t=3200 s) indicates these constructs binding their cytokines very tightly, much more so than unmodified mAb or other polysaccharide-mAb constructs investigated.

FIG. 9 shows binding constants (K_{D}) measured and calculated using ForteBio Octet System. The results are shown as the average of the five separated measurements of each condition, and the error bars are shown as the standard deviation. (*) p<0.001.

FIG. 10 shows an imaging cytometry result of PMA-differentiated THP-1 macrophages stained with p65 subunit of nuclear factor-kappa B (NF-κB) and nucleus after stimulated by various different conditions. (A) Representative imaging cytometry image of THP-1 macrophages treated with 100 ng/ml of IL-1β. The nuclei appear as bright spots surrounded by bright rings, which represents the stained cytosolic NF-κB. (B-E) Histogram representation of the NF-κB translocation value of THP-1 macrophages treated with (B) culture media, (C) 100 ng/ml IL-1β, and (D) CMC-anti-IL-1β+100 ng/ml IL-1β. A dashed line represents the 90^{th} percentile of the unstimulated THP-1 macrophage population, and this translocation value of 320 was used as a threshold to identify responsive THP-1 cells in the stimulated populations. The number beside the dashed line in each histogram is the fraction of stimulated cells determined by the threshold.

FIG. 11 shows a fraction of the THP-1 cells with translocation values above the threshold defined as the 90^{th} percentile in the unstimulated control group. (i) 100 ng/ml LPS, (ii) culture medium, (iii) IL-1β, (iv) 0.1 wt % CMC-anti-IL-1β+100 ng/ml IL-1β, (v) 0.1 wt % HA-anti-IL-1β+100 ng/ml IL-1β, (vi) 100 μg/ml anti-IL-1β+100 ng/ml IL-1β, (vii) 100 ng/ml TNF-α, (viii) 0.1 wt % CMC-anti-TNF-α+100 ng/ml TNF-α, (ix) 0.1 wt % HA-anti-TNF-α+100 ng/ml TNF-α, (x) 100 μg/ml anti-TNF-α mAb+100 ng/ml TNF-α, (xi) CMC, and (xii) HA. Error bars represent the standard deviation of four separate samples.

FIG. 12 shows neutralization of IL-1β or TNF-α′ signaling by alginate-(anti-IL-1β) or alginate-(anti-TNF-α), respectively, as compared to unconjugated mAb, LPS, or non-stimulated Control. Alginate appears to induce some NF-κB translocation compared to non-stimulated Control, but the alginate-mAb conjugates still provide comparable neutralization as unmodified mAb. Error bars represent the standard deviation of four separate measurements.

FIG. 13 shows histology sections showing the effects of alginate solution delivery (left) or (anti-TNF-α)-alginate delivery (right). Sites treated with alginate showed early signs of wound healing with new connective tissue forming whereas sites treated with (anti-TNF-α)-alginate showed signs of inhibited wound healing, characterized by a lack of dense tissue at the delivery site.

### DETAILED DESCRIPTION OF THE INVENTION

Carboxymethylcellulose (CMC) is a derivative of cellulose, which is a polysaccharide composed of D-glucose. The biological activity of cellulose has also been extensively explored, and the implantation of CMC showed no signs of material-induced inflammation or host rejection, indicating that CMC is biocompatible and immunologically inert. CMC also has other attractive properties for conjugating therapeutics, providing ease of modification and delivery in aqueous media. High molecular weight CMC (defined here as greater than 100 kDa) is more resistant to degradation than HA when implanted. So in addition to its lack of biological activity compared to HA, it also provides a method for sustained delivery.

Alginate is an anionic polysaccharide derived from seaweed. It binds calcium cations avidly, and calcium-crosslinked alginate gels have been showed to be chemically and immunologically inert in vivo. The molecular weight of commercial formulations can be up to 600 kDa, and solutions derived from these are highly viscous.

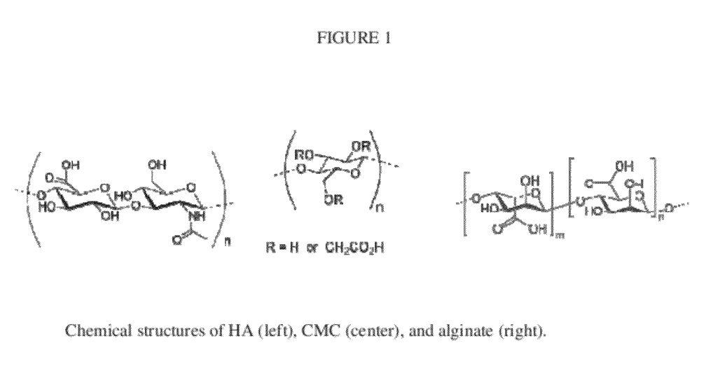

The monomer structures of HA, CMC, and alginic acid (referred to here as alginate) are shown in FIG. 1. For the given structures, n may be in the range of 1-100,000 for all these polysaccharides. In HA, every other cyclic sugar has a carboxylic acid group that is potentially negatively charged at neutral pH, making the effective degree of anionic functionalization 0.5. The degree of cellulose carboxylation depends upon treatment conditions. One type that may be used in embodiments presented herein has a degree of anionic functionalization equal to 0.9 (90% of monomers had one carboxylic acid group attached). A diversity of chemical strategies may be used to modify the material or biochemical properties of the final products. For alginate, both the β-D-mannuronate and the α-L-guluronate monomers have a carboxylic acid group, making the degree of anionic functionalization equal to 1.0. The charge density of alginate may play a role in its contribution to binding affinities.

We have conjugated anti-tumor necrosis factor-α (TNF-α) and anti-interleukin-1β (IL-1β) monoclonal antibodies to high-molecular weight HA and demonstrated the neutralizing activities of these cytokines both in vitro and in vivo. This is reported in International Patent Application Publication No. WO2009/026158, which is incorporated by reference herein. Significant reductions in the numbers of invading macrophages and shifts in their phenotypes in a rat incisional wound model were observed following treatment with 50 μg mAb, compared to standard systemic doses in humans of 100 mg. This approach has the potential for increased antibody activities at significantly lower doses and could be more effective at neutralizing cytokine activities.

Polyclonal antibodies (Raponi, et al. (1993) “Differential effect of human and murine polyclonal and monoclonal antisera on TNF-alpha production by human monocytes” J. Chemother. 5, 317-324), antibody fragments (Chen, W., et al, (2009) “Improved isolation of anti-rhTNF-alpha scFvs from phage display library by bioinformatics” Mol. Biotechnol. 43, 20-28), peptides (Takasaki, et al. (1997) “Structure-based design and characterization of exocyclic peptidomimetics that inhibit TNF alpha binding to its receptor” Nat. Biotechnol. 15, 1266-1270), aptamers (Guthrie, et al. (2006) “Assays for cytokines using aptamers” Methods 38, 324-330), and other cytokine-binding moieties could also be incorporated into the types of conjugates described here. Constructs composed of chemically modified alginates as well as other polysaccharides besides alginates may also be used in preparing these constructs. These polysaccharides include, for example, but are not limited to chitosan, fucoidan, dextran and derivatives such as dextran sulfate, pentosan polysulfate, carrageenans, pectins and pectin derivatives, and cellulose derivatives. Synthetic polymers containing charged functional groups may be used instead of polysaccharides to exhibit the types of binding effects observed in these polysaccharide conjugates, such as poly(acrylic acid), poly(methacrylic acid), poly(acrylamide), charged polystyrene derivatives, polyvinylpyrrolidone.poly(amino acids), poly(amines), and other polyelectrolytes.

Reactions that could be adapted for performing the conjugation between antibodies and polysaccharides include, for example, Michael-type additions (Oh, et al., (2008) “Signal transduction of hyaluronic acid-peptide conjugate for formyl peptide receptor like 1 receptor” Bioconjug. Chem. 19, 2401-2408), disulfide bond formation (Liu, et al. (2004) “Disulfide-crosslinked hyaluronan-gelatin sponge: growth of fibrous tissue in vivo” J. Biomed. Mater. Res. A. 68, 142-149), click reactions (Malkoch, et al., (2006) “Synthesis of well-defined hydrogel networks using click chemistry” Chem. Commun. (Camb), 2774-2776) formation of a Schiff base and amide-bond formation described in this application. Some of these may require prior chemical functionalization of the antibody, polysaccharide, or both. See Bhargava, et al. (1999). “Synthesis of aminobenzyltriethylenetetraminohexaacetic acid: conjugation of the chelator to protein by an alkylamine linkage” J. Protein Chem. 18, 761-770.

Inflammatory mediators such as the cytokines play a central role in determining the balance of biochemical factors at the sites of injury or disease. Cytokines are regulatory peptides that can be produced by every nucleated cell type in the body. Cytokines have pleiotropic regulatory effects on haematopoietic and many other cell types that participate in host defense and repair processes. Cytokines therefore include lymphocyte-derived factors known as ‘lymphokines’ monocyte-derived factors call ‘monokines,’ haematopoietic ‘colony-stimulating factors,’ connective tissue ‘growth factors,’ and chemotactic chemokines. (Thomson, A. W., and Lotze, M. T., eds. (2003) “The Cytokine Handbook, 4th edn” (London: Academic Press). Pro-inflammatory mediators, such as the cytokines IL-1β, IL-6, and TNF-α, induce the expression of several collagenases, including MMP-1, MMP-2, and MMP-9, which promote the chronic non-healing wound state. Elevated MMP expression due to increased inflammatory mediator levels further perpetuates the chronic wound state by proteolytically inactivating important growth factors such as PDGF and vascular endothelial growth factor (“VEGF”). Furthermore, TNF-α and TGF-β synergistically promote the production of MMP-9 by fibroblasts. Pro-inflammatory mediators also inhibit collagen synthesis in cultured fibroblasts, effectively inhibiting the deposition of new tissue.

The production of cytokines and other pro-inflammatory mediators is often autoinductive, and exposure to a low concentration of mediators can lead to a signaling cascade and the production of more mediators. For example, chronic wounds appear to lack mechanisms capable of controlling mediator production, resulting in an imbalance in their concentrations. Therefore, restoring this balance is an effective approach for treating inflammatory conditions. When treated with a composition as described herein that inhibits the activity of pro-inflammatory mediators, such as IL-1β, IL-6, and TNF-α, or TGF-β activity, a concomitant reduction in neutrophil invasion is expected, coupled with an increase in rate of healing.

In one general aspect, the various embodiments of the invention are directed to a composition including a hydrophilic polymer and a ligand binding moiety covalently attached to the polymer. Suitable hydrophilic polymers may include, for example, but are not limited to polysaccharides, such as chitosan, fucoidan, dextran and derivatives such as dextran sulfate, pentosan polysulfate, carrageenans, pectins and pectin derivatives, and cellulose derivatives. Synthetic polymers containing charged functional groups may be used instead of polysaccharides to exhibit the types of binding effects observed in these polysaccharide conjugates, such as poly(acrylic acid), poly(methacrylic acid), poly(acrylamide), charged polystyrene derivatives, polyvinylpyrrolidone.poly(amino acids), poly(amines), and other polyelectrolytes. Suitable ligand binding moieties may include, for example, but are not limited to monoclonal antibodies, polyclonal antibodies, antibody fragments, phage-derived peptides, soluble receptors and receptor fragments, and nucleic acid-based aptamers. Covalent attachment may be effected, for example, by Michael-type additions, disulfide bond formation, click reactions, formation of a Schiff base, and amide-bond formation described in this application.

In one embodiment a specific composition that is shown to provide enhanced binding of anti-IL-1β and anti-TNF-α for their cytokines when conjugated to sodium alginate.

These conjugates may display synergistic binding for the target cytokine, wherein the polysaccharide conjugate is directly involved in the binding, or the conjugate may promote re-binding, wherein dissociated cytokine is retained in close proximity to the antibody, which facilitates a subsequent binding event. This results in a significant change in the association or dissociation kinetics and a resultant enhancement of the equilibrium binding constant. This effect may be quite general, including alginate conjugated to polyclonal antibodies, antibody fragments, peptides, and DNA- or RNA-based aptamers. It may also extend to conjugation of chemically modified alginates as well as other polysaccharides besides alginates.

Conjugation of mAb against the pro-inflammatory cytokines IL-1β or TNF-α to high molecular weight HA or CMC results in conjugates that retain their cytokine binding affinities. Changes in K_{D} were observed with different pairs of polysaccharide and cytokine, suggesting that cooperative effects due to the biopolymer influence the interactions between mAb and cytokine. This effect appears to be particularly strong in the alginate-mAb conjugates measured in these studies. Cell assays of the neutralization of cytokine signaling by these constructs confirm their cytokine-binding functions in dilute solution. In a setting of local inflammation, we suggest that these constructs could represent a novel method of delivering cytokine-neutralizing antibodies.

The following examples are directed to various embodiments of the invention.

### Example 1

### Preparation of Materials

Hyaluronic acid (HA, M_{w}˜1.6 MDa), carboxymethyl cellulose (CMC, M_{w}˜700 kDa) with 90% carboxylation per CMC monomer (provided by manufacturer), sodium alginate (M_{w}˜600 kDa), glycidyl methacrylate, N-hydroxysulfosuccinimide sodium salt (sulfo-NHS), N-(3-dimethylaminopropyl)-N′-ethylcarbodiimide hydrochloride (EDC), and 4-(dimethylamino)pyridine (4-DMAP) were purchased from Sigma-Aldrich (St. Louis, Mo.) and used as received. Anti-hIL-1β and anti-TNF-α, both from purified mouse monoclonal IgG1, were purchased from R&D Systems, Inc. (Minneapolis, Minn.) with reported binding affinities for their cytokines of 120 pM. Recombinant human IL-1β (IL-1F2) and human TNF-α were also purchased from R&D Systems, Inc. All reagents were reconstituted and stored according to the manufacturer's instructions.

### Example 2

### Polysaccharide-mAb Preparation

The first step reaction consisted in the activation of the carboxylic acid in HA, CMC, or alginate. The activated ester intermediates were subsequently used as precursors for the coupling reaction with anti-IL-1β or anti-TNF-α. First, 10 mg of polysaccharide was dissolved in phosphate buffer saline (pH˜7.4) (2 ml). EDC (120 μg, 625 nmol), sulfo-NHS (217 μg, 1 μmol), and 4-DMAP (10 μg) were added as solids to the solution and allowed to dissolve and react overnight before adding mAb. The polysaccharide-NHS active ester is a versatile precursor for bioconjugation with primary amines. Antibodies (0.3 mg) were added to the activated HA solution, and the reaction proceeded at room temperature for 16 h. The solution was then twice precipitated in a saturated solution of ammonium sulfate, and the product was recovered by centrifugation. The pellet was reconstituted in PBS and followed by dialysis (Nest Group, Southborough, Mass.) against pure PBS for 4 hr (MW cut-off 300 kDa).

### Example 3

### Polyacrylamide Gel Electrophoresis

A 10 mL solution of 4% acrylamide/bis-acrylamide in 1% TBE buffer was prepared from 40% acrylamide/bis-acrylamide (Sigma) and 10×TBE buffer (Promega, Madison, Wis.). The solution was mixed on a stir plate for 10 min, and followed by adding 50 μl of 10% (w/v) ammonium persulfate and 4 μL of N,N,N′,N′-tetramethylethylenediamine (Sigma). The solution was mixed well and injected into glass plates (Bio-Rad, Hercules, Calif.). The gel set after 30-60 mins. Then 5 μL of each of the standards consisting of 0.1% HA, 0.05% HA, 0.025% HA in the gel were loaded at two different concentrations using 1× and 0.1× stock solutions, and 125V used to electrophorese the gel for 5 h. Gels were stained in 0.5% Alcian Blue (Sigma) with 3% acetic acid for 45 min followed by destaining with 3% acetic acid overnight. Gel images were taken and quantitatively analyzed using Fujifilm LAS-3000 and MultiGauge image analysis software.

### Example 4

### Fluorescence Immunosorbent Assay

Immuno 96 MicroWell Plate (NUNC, Rochester, N.Y.) was first incubated with 50 μl of 1 μg/ml of rabbit Anti-Rat IgG (Jackson, Pa.) in PBS each well at 4° C. overnight. The solution was removed and the plate was washed three times with detergent followed by incubation at 37° C. for 1 h in 200 μl of the blocking buffer, which contained 0.25% BSA, 0.05% Tween, and 1 mM EDTA in 1×PBS. The plate was subsequently washed. Interleukin-1β antibody was dissolved in carbonate buffer, and standards were prepared using rat whole IgG (Jackson, Pa.) in triplicates. 50 μl of each solution was loaded into designated wells followed by one hour of incubation with shaking at room temperature. The solutions were discarded and each well was washed in detergent three times with ten minutes of incubation in between. 2 μg/ml of goat anti-rat IgG conjugated with Alexa 488 (Invitrogen, CA) was prepared in carbonate buffer. 50 μl of this solution was loaded into each well followed by one hour incubation with shaking in the dark at room temperature. Wells were washed again three times with detergent for 10 min in between and the plates preserved with PBS. The plate was read and analyzed by SAFIRE microplate reader with excitation at 488 nm and emission at 520 nm.

### Example 5

### Binding Affinity Measurements

The Octet system (ForteBio Corp.) was utilized to measure binding affinities of unmodified and conjugated mAb for their cytokines. The Octet measures the reflection coefficient as broadband visible light propagates to the end of a fiber optic. Changes in the refractive index at the fiber optic-solution interface result in a wavelength-dependent shift in the maximum of the reflection coefficient. Association and dissociation curves are fit to equations of the following form:

R(t)=R₀+ΔR{1−exp[−kₒₙ(t−t₀)]} Eq. 1

R(t)=R₀+ΔRexp[−k_{off}(t−t_{off})] Eq. 2

where R(t) is the reflection coefficient at time t, R₀ is the baseline value of the reflection coefficient, ΔR is the total change in response, kₒₙ is the association rate constant, tₒₙ the time at which the sensor is placed in solution containing the analyte, k_{off} is the dissociation rate constant, and t_{off} is the time at which the sensor is placed in pure buffer solution. The values for kₒₙ and k_{off} are determined from curve fitting, and their ratio provides a measurement of K_{D}. Protein A sensor tips were hydrated in sample diluent (0.02% Tween 20, 150 mM NaCl, 1 mg/ml BSA, 10 mM phosphate buffered saline, and 0.05% sodium azide) supplied by ForteBio for at least five minutes before the experiment. All the samples were diluted in buffer: mAb were diluted to 10 μg/ml, and the polysaccharide-mAb samples were diluted to the equivalent concentration. Cytokines were diluted to desired concentration in sample diluent. The experimental setup is as followed in the following specific sequence: sample diluent 5 minutes (baseline), mAb or polysaccharide-mAb solution 60 min (loading), sample diluent 10 min (wash), sample diluent 10 min (wash), sample diluent 15 min (baseline), cytokine solution 40 min (association), and sample diluent 60 min (dissociation). The results were analyzed by the ForteBio analysis program that generated the best-fit binding isotherm and the association rate kₒₙ and dissociation rate k_{off} were calculated from the isotherm.

### Example 6

### Imaging Cytometry

THP-1 human acute monocytic leukemia cells were cultured at concentrations between 0.5-7×10⁵ cells/ml in RPMI 1640 (Cellgro, Va.), containing 10% FCS, L-glutamine, 100 U/ml Penicillin, and 100 μg/ml Streptomycin, and maintained at 37° C. in 5% CO₂. 15,000 THP-1 cells were plated onto each well of a 96-well plate. Each well was treated with media containing 20 nM of PMA for 48 h at 37° C. followed by 24 h of recovery in fresh media before analysis.

Cells were stimulated according to each experimental condition for 30 minutes. The supernatants were gently aspirated and stimulated cells were fixed with 2% paraformaldehyde in plates for 10 min. They were then subsequently washed once in PBS and permeabilized for 10 min with permeablizing buffer. They were incubated in the presence of 1 μg/ml anti-NF-|B antibodies for 1 hr. Cells were treated with detergent for 10 min followed by 1 hr incubation in the presence of 2 μg/ml secondary antibodies and 1 μg/ml solution of Hoechst 33342. Cells were then treated with detergent for 10 min and 200 μl of PBS were added to each well. Plates were covered and stored at 4° C. until analysis.

Data Acquisition and Analysis: The ArrayScan VTI imaging cytometer (Cellomics, Pittsburgh, Pa.) is an automated fluorescent imaging microscope that acquires spatial information of the fluorescently labeled cell surface, organelles, or cytosol in cells. The system scans a designated number of several fields in each individual well. Analysis of the images of each well was conducted according to a predefined algorithm. The system acquired images of the fields in each well until a predefined number of cells been identified and analyzed. The ArrayScan was equipped with emission and excitation filters for different fluorescent signals (Omega, Brattleboro, Vt.) emitted by Hoechst 33342, Alexa 488, and Alexa 680. Data were acquired and analyzed by ArrayScan Compartmental Analysis Bioapplication version 5.5.1.3 and HCS viewer (Cellomics). The Compartmental Analysis Bioapplication Software measures the average and total fluorescence intensity of specified subcellular regions, in this case the nucleus and cytosol. In our experiments, the software measured the average fluorescence intensity of NF-κB specific stain inside the nucleus. This value is subtracted from the mean fluorescent intensity of NF-κB in the cytosol to generate a mean value of translocated protein. That is used to subtract the average fluorescence intensity of NF-κB specific stain in specified donut ring around the nucleus. The difference demonstrated a measure of amount of NF-κB translocation, which was an indication of the level of cellular inflammatory response.

### Example 7

### Animal Studies

All animal experiments were performed following the policies and procedures of the Institutional Animal Care and Use Committee at the University of Pittsburgh. Incisional wounds were created in healthy adult Sprague-Dawley rats to evaluate the effect of the samples upon the local acute inflammatory response of the host. Four separate 1-cm incisions were made on the backs of shaved, anesthetized animals, and each was used as a treatment or control site for these experiments. A total of 12 rats were used in this study; all treatment regiments were repeated at least 5 times. The underlying fascia was scraped with a scalpel to accentuate tissue damage. The site was then treated with alginate solution or solution of (anti-TNF)-alginate conjugate then sutured shut. After 4 days, the animals were sacrificed and the sites were collected for histological analysis. This time point was chosen because it is roughly in the middle of the inflammation phase of acute wound healing but long enough from initial injury to provide a preliminary measure of the persistence of the anti-inflammatory effects of the gels.

### Example 8

### Histological Assessment of Wound Site

The explanted wound site specimens were mounted on glass slides and embedded in paraffin, then subsequently deparaffinized with xylene followed by a graded series of ethanol solutions (100-70%). Sections were then stained with Masson's Trichrome using a microwave staining protocol (Richard-Allen Scientific, Kalamazoo, Mich.) for morphological assessment.

### Example 9

### HA-mAb and CMC-mAb Composition

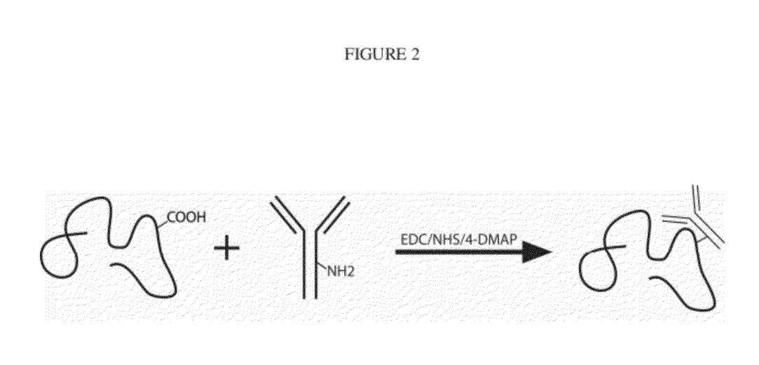

Monoclonal IgG antibodies that were capable of neutralizing the pro-inflammatory cytokines TNF-α and IL-1β were separately conjugated to HA and CMC using carbodiimide coupling chemistry, as shown in FIG. 2. Carboxyl groups on HA and CMC were first activated by sulfo-NHS through EDC-mediated chemical reaction. The antibodies were then introduced into the solutions of activated HA and CMC. The primary amine groups on the antibodies reacted with NHS to form amide linkage, and the samples were purified by ammonium sulfate precipitation followed by dialysis using a 300 kDa-cutoff membrane to remove both residual chemical impurities as well as unconjugated antibody.

Polysaccharides were partially activated at carboxylic acid moieties followed by coupling reaction with pendant amines on the mAb (H₂N-mAb). Carboxymethylcellulose (CMC) and hyaluronic acid (HA) were used as two representative polysaccharides to compare to alginate conjugates.

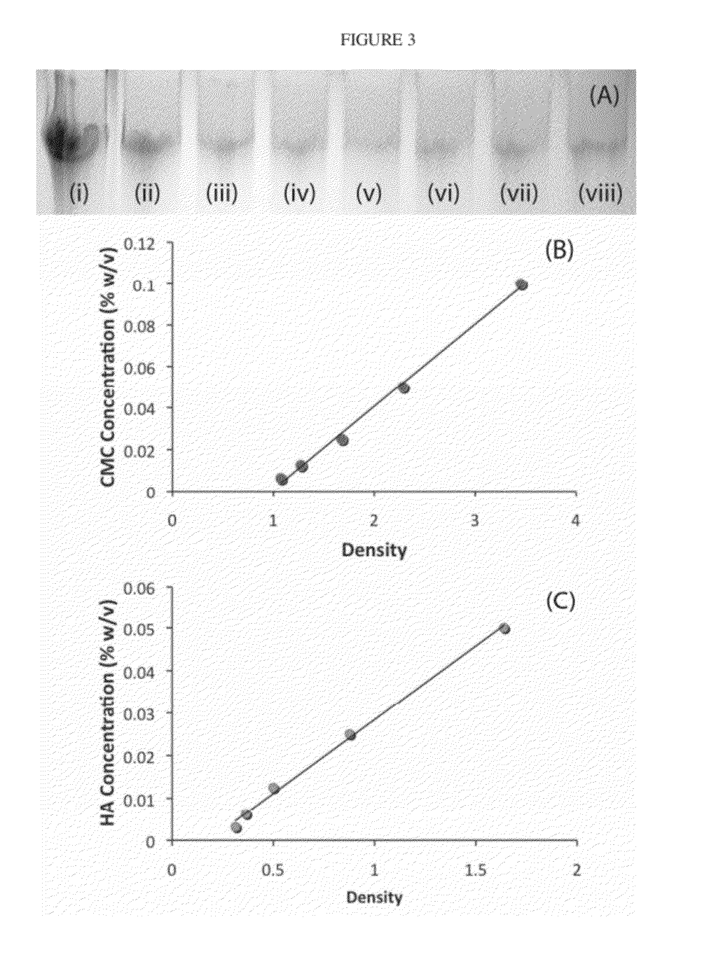

To measure the composition of the conjugates, the concentrations of polymer and antibodies were measured separately. The method to measure the concentration of HA using non-denaturing PAGE was described previously by Sun et al., and the same 4% gel was applied to measure the concentration of CMC. A representative gel is shown in FIG. 3A. The integrated intensities of the bands were correlated with the serially diluted CMC standards to generate a calibration curve, shown in FIG. 3B, and those from HA standards are shown in FIG. 3C. Due to the high molecular weight nature of CMC, the gel was saturated at CMC concentrations of 0.2% and excessive spreading of the bands was observed. In order to accurately estimate the concentration of CMC in the conjugate, highest concentration was determined to be 0.1% (w/v), and all the samples were diluted 1:10 to fit in the range of calibration curve. Results are tabulated in Table 1.

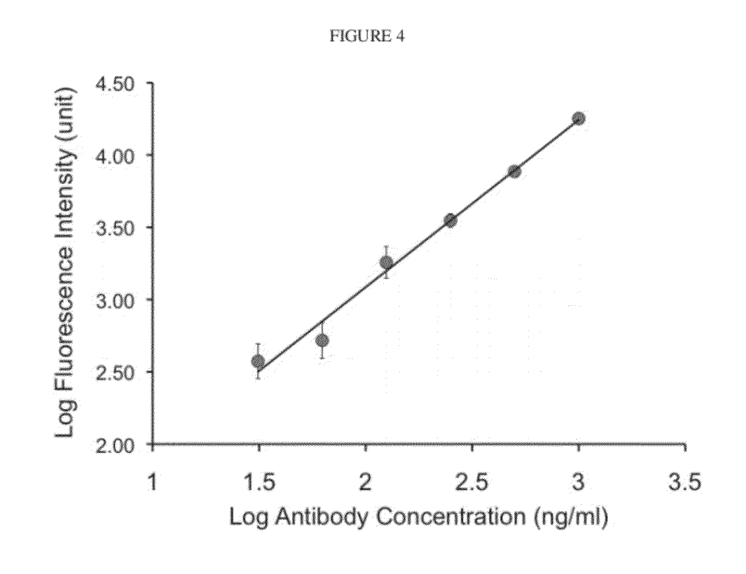

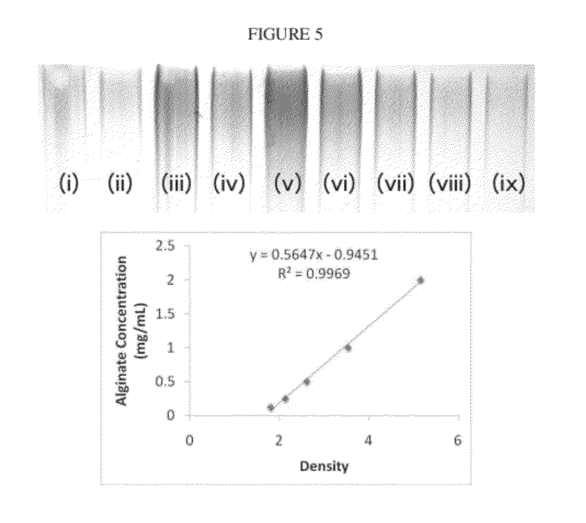

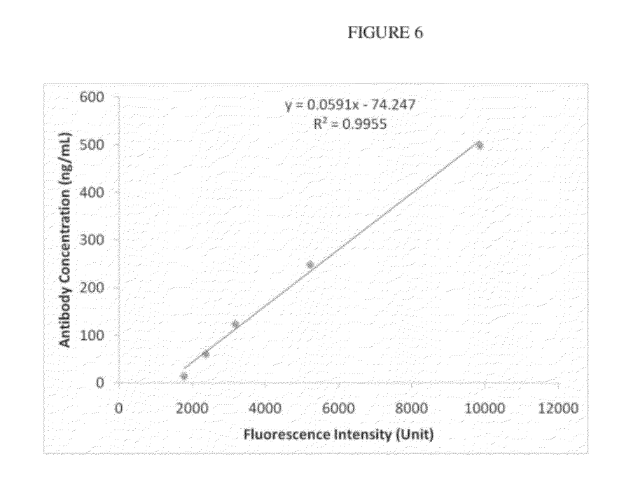

Sandwich fluorescence immunosorbent assays were utilized to quantitatively determine the concentration of monoclonal antibody in the conjugates. A calibration curve was generated using rat whole IgG, and the results are shown in FIG. 4. The concentrations of the antibodies in the different conjugate systems are shown in Table 1, as well as the ratios of mAb to polysaccharide. The molar ratios of mAb to CMC are significantly lower than those of mAb to HA, probably due to two factors: the degree of carboxylation and molecular weight of CMC. CMC, unlike HA, does not have carboxylic acid group on each of the repeat units, so the coupling reaction is likely to be less efficient for CMC than HA. However, the main factor was that the molecular weight of CMC was approximately 10% that of HA in this study, and hence there were more polysaccharide chains in a given CMC solution than a HA solution with the same concentration by mass. Using these methods, as shown in FIGS. 5 and 6, the ratio of mAb:alginate was measured to be 3.8%, which is comparable to the CMC and HA conjugates.

TABLE 1
Summary of compositions determined from PAGE
and fluorescence immunosorbent assays.

| | | | mAb: |
|---|---|---|---|
| | [Polysaccharide] | [Antibody] | Polysaccharide |
| | (μM) | (μM) | (mol %) |
| HA-antiIL-1β | 0.773 | 0.481 ± 0.026 | 62.23 ± 3.37 |
| HA-antiTNF-α | 1.209 | 0.437 ± 0.058 | 36.15 ± 4.80 |
| CMC-antiIL-1β | 7.367 | 0.530 ± 0.043 | 7.19 ± 0.588 |
| CMC-antiTNF-α | 9.200 | 0.594 ± 0.091 | 6.46 ± 0.986 |

Separate measurements of polysaccharide and antibody concentrations provide the basis for determining the degree of functionalization in polysaccharide:mAb conjugates.

### Example 10

### Cytokine Binding Affinity of HA-mAb and CMC-mAb Conjugates

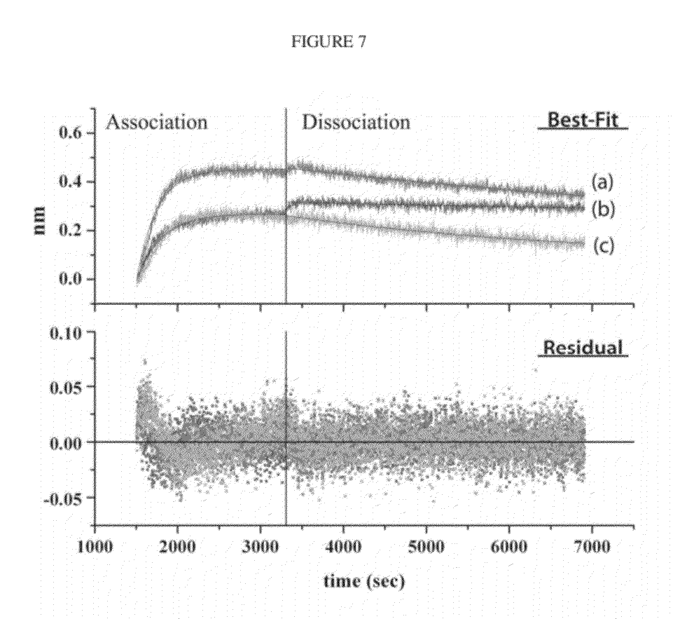

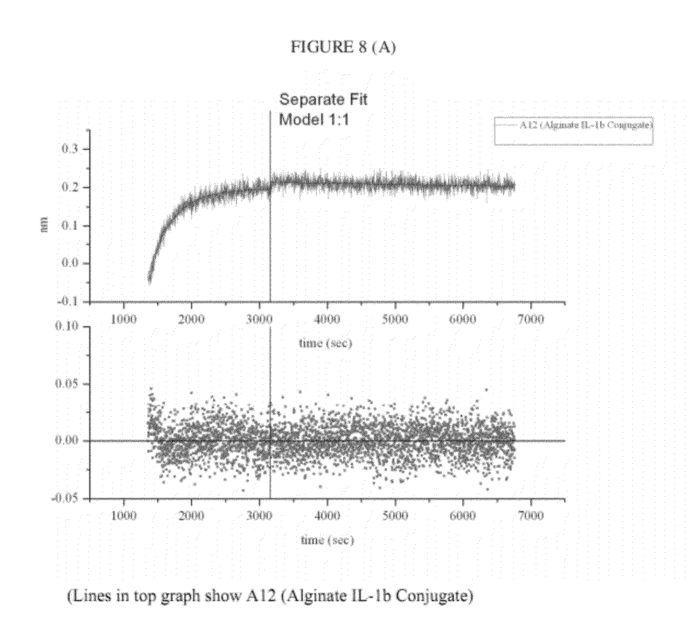

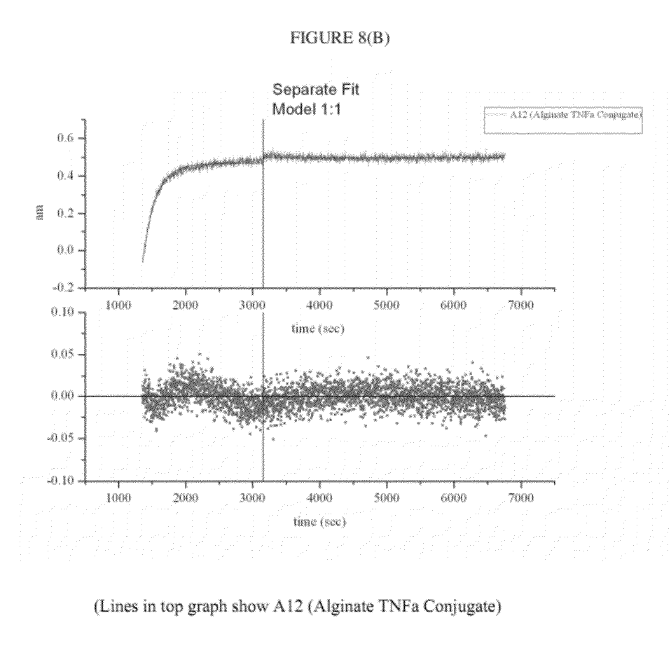

The binding constants of the four individual conjugates of polysaccharide and mAb were measured and compared to those of the non-conjugated anti-IL-1β and anti-TNF-α mAb. All mAb were biotinylated to bind strongly with streptavidin-functionalized sensor tips. The rise in signal, shown in FIG. 7, demonstrated the change in refractive index at the sensor-solution interface, indicating that the cytokines were interacting with the sensor surface coated with antibodies or conjugates. After the signal reached saturation, the sensors were immersed in buffer solution to measure the dissociation of the cytokines from sensor surface. The association and dissociation curve were fit by Equation 1 and 2 respectively to generate isotherms and obtain the corresponding binding constants. The standard deviations of the binding constants were calculated based on three separate measurements. Although the interaction between streptavidin and biotin was very strong, a slight drifting baseline was still observed due to slow dissociation of this non-covalent interaction. Therefore, association and dissociation isotherms were corrected with corresponding baseline to obtain accurate results.

The results from affinity binding experiment are shown in Table 2. The K_{D} for both anti-IL-1β was measured to be 118.0±19.5 pM while that for HA-(anti-IL-1β was 40.13±4.79 pM and CMC-(anti-IL-1β was 412.2±1.30 pM. The association kinetics were essentially identical, with kₒₙ values of (6.548±0.384)×10⁵/Ms for non-conjugated anti-IL-1β, (6.247±0.228)×10⁵/Ms for HA-(anti-IL-1β), and (5.588±0.595)×10⁵/Ms for CMC-(anti-IL-1β). The main factor contributing to differences in K_{D} for the conjugates compared to the non-conjugated mAb appeared in the dissociation kinetics: IL-1β dissociated three-times more slowly from the HA-(anti-IL-1β) but three-times faster from CMC-(anti-IL-1β). This suggests that neither HA nor CMC interferes with the formation of the antigen-antibody complex, but HA appeared to stabilize this complex somewhat while CMC appeared to destabilize it. The exact mechanisms for these interactions are not clear, but the conjugation of high molecular weight polysaccharides to anti-IL-1β can influence the binding kinetics. However, in both constructs, IL-1β binding was still observed.

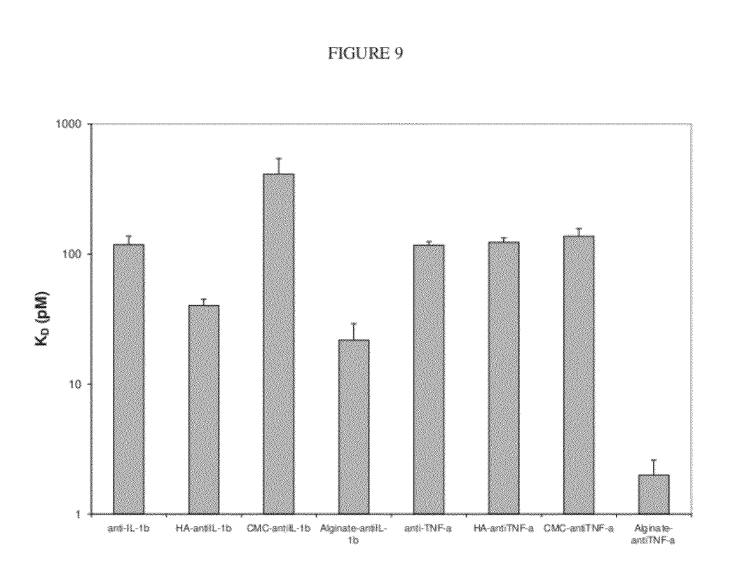

Non-conjugated anti-TNF-α was measured to have an equilibrium binding constant of (117.2±7.34) pM, also consistent with manufacturer's specifications. Both the HA-(anti-TNF-α) and CMC-(anti-TNF-α) conjugates had similar values of K_{D}; (123.0±10.0) pM and (137.4±20.0) pM, respectively. However, in both HA and CMC, the rate constants kₒₙ and k_{off} were approximately two-times slower, leaving the ratio of k_{off} to kₒₙ similar but suggesting that adsorption and desorption of TNF-α to the constructs were slightly inhibited. TNF-α is a trimeric protein with a molecular weight that is three-times greater than that of IL-1β, which may contribute to the differences in kinetics between conjugated and non-conjugated mAb. In binding anti-TNF-α, though, both polysaccharide conjugates appear to have good binding affinity. The results are summarized in FIG. 9 and Table 2 for both anti-IL-1β and anti-TNF-α constructs.

When conjugated to alginate, both anti-IL-1β and anti-TNF-α′ demonstrated significantly stronger binding affinities for their cytokines, measuring 21.8±7.354 pM and 2.027±0.612 pM, respectively. This was primarily due to large decreases in k_{off}, which could be attributed to cooperative binding or enhanced re-binding by the alginate-mAb conjugate.

TABLE 2
Summary of binding constants describing interactions between
mAb and polysaccharide-mAb conjugates with the cytokines.

| | kₒₙ (10⁵ 1/Ms) | k_{off} (10⁻⁵ 1/s) | K_{D} (pM) |
|---|---|---|---|
| Anti-IL-1β mAb | 6.548 ± 0.384 | 7.906 ± 1.683 | 118.0 ± 19.5 |
| HA-anti-IL-1β | 6.247 ± 0.228 | 2.505 ± 0.320 | 40.13 ± 4.79 |
| CMC-anti-IL-1β | 5.588 ± 0.595 | 24.44 ± 7.758 | 412.2 ± 1.30 |
| Alg-anti-IL-1β | 5.493 ± 0.443 | 1.177 ± 0.311 | 21.8 ± 7.354 |
| Anti-TNF-α mAb | 6.448 ± 0.154 | 7.558 ± 0.468 | 117.2 ± 7.34 |
| HA-anti-TNF-α | 13.60 ± 1.08 | 16.66 ± 0.924 | 123.0 ± 10.0 |
| CMC-anti-TNF-α | 9.874 ± 0.610 | 14.16 ± 1.54 | 137.4 ± 20.0 |
| Alg-anti-TNF-α | 5.493 ± 0.443 | 0.174 ± 0.501 | 2.027 ± 0.612 |

### Example 11

### In Vitro Cytokine Neutralization by Polysaccharide-mAb Conjugates

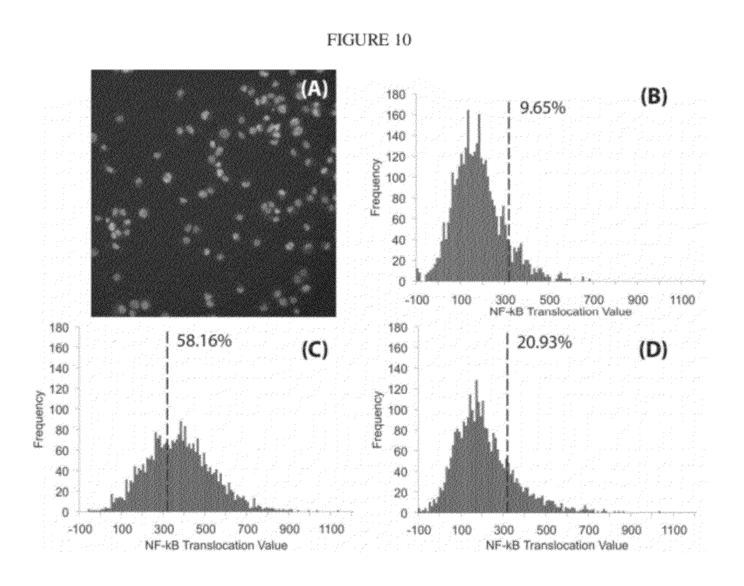

In order to validate the biological activities of these conjugates, THP-1 human acute monocytic leukemia cells were differentiated into macrophages and exposed to solutions containing IL-1b or TNF-α with or without polysaccharide-mAb conjugates. Measuring the translocation of cytosolic NF-κB into the nucleus using imaging cytometry provides a method to quantify incipient inflammatory responses, and images used for analysis are shown in FIG. 10A. The compartment analysis protocol quantitatively measured and compared the amount of NF-κB stained in the nucleus and cytoplasm. The difference of the amounts of NF-κB measured in these two compartments was used as an indicator of the level of inflammatory stimuli. Increases in the translocation value are expected following exposure to pro-inflammatory cytokines, and lower levels should be observed if cytokines are neutralized by mAb in solution.

Cells were classified as stimulated or unstimulated with an arbitrary threshold of the 90^{th} percentile of the NF-κB translocation value in the negative control samples. Representative histograms are shown in FIG. 10B-D. In FIG. 10B is shown the histogram of translocation values for unstimulated cells, with the most probable value of 130 and the 90^{th} percentile at 320. In cultures stimulated with IL-1β, 58% of the cells had translocation values above 320, as shown in FIG. 4C, while cultures with IL-1β and CMC-(anti-IL-1β) had 21% of the cells with translocation values above threshold. This suggests that CMC-(anti-IL-1β) is effective at reducing the signaling of IL-1β in solution.

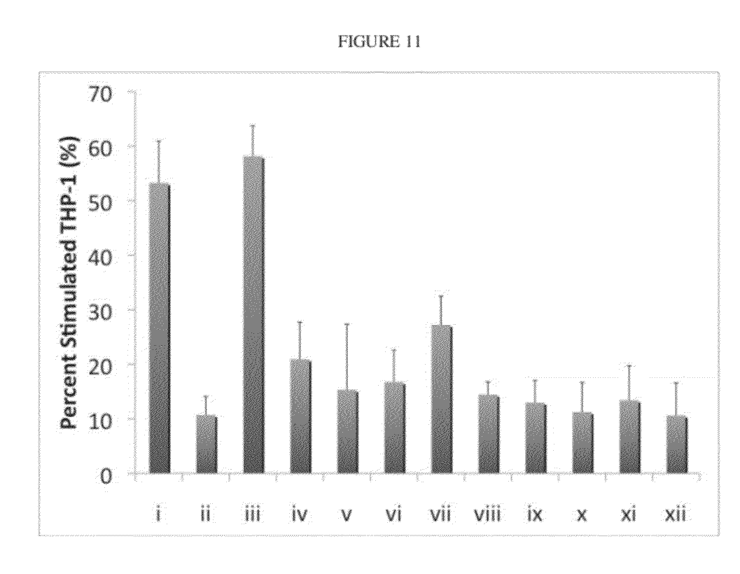

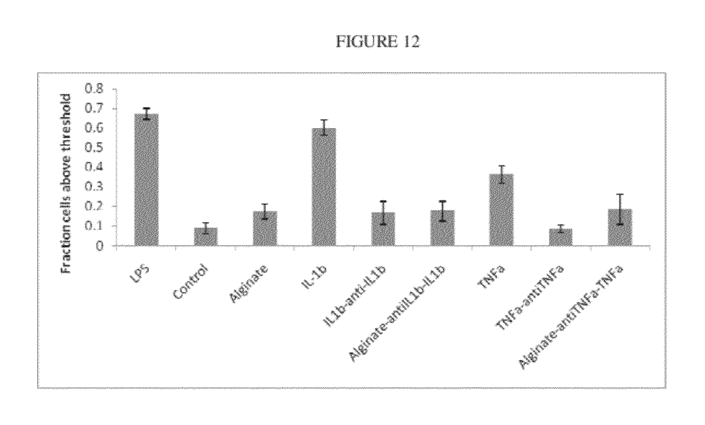

The results of histogram analyses across the different conditions are shown in FIG. 11. When stimulated with LPS, 53.3±7.7% of cells had translocation values above threshold while 58.2±5.6% of those stimulated with IL-1β were above threshold and 27.2±5.2% when stimulated with TNF-α. Treatment with solutions containing IL-1β/anti-IL-1β and TNF-α/anti-TNF-αresulted in translocation values of 16.7±5.3% and 14.4±6.4%, respectively. This indicates that antibodies are binding the cytokines and preventing most, but not all, NF-κB activation from occurring. For all compositions of CMC-mAb and HA-mAb conjugates (shown in FIG. 11) and alginate-mAb conjugates (shown in FIG. 12), similar ranges of the translocation values were observed for neutralizing the effects of IL-1β or TNF-α, which indicates that the polysaccharide constructs have comparable or better cytokine-neutralizing activities as unmodified mAb.

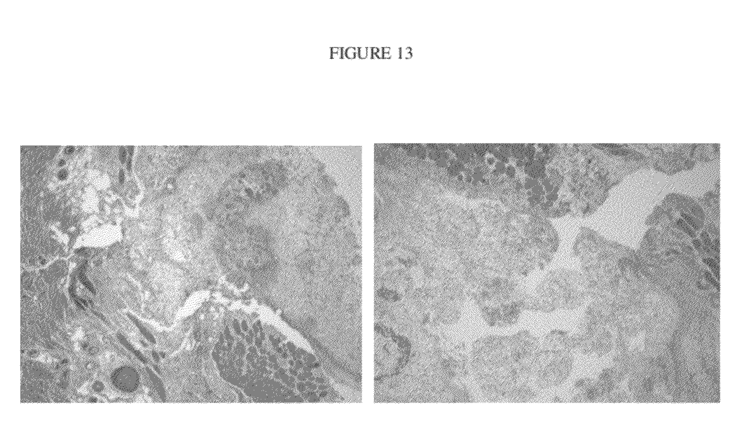

The (anti-TNF-α)-alginate conjugate was tested in a rat incision wound model and compared to the effects of delivery of alginate in buffer solution. In FIG. 13 are shown histology images at Day 2 following injury and delivery of the cytokine inhibitor or control. The density of tissue observed at the injury site is much lower where the TNF-α inhibitor was delivered, suggesting that early wound healing processes have been inhibited through the actions of the cytokine inhibitor.

Documents cited herein are incorporated by reference and may be indicative of the states of the art and/or may be useful in understanding certain aspects of the invention when read in the context of this disclosure. Their inclusion here is not an admission that they are prior art for any purpose.

I claim:

1. A composition comprising:
a cytokine-inhibiting molecule; and
a polysaccharide, wherein the cytokine-inhibiting antibody is covalently attached to the polysaccharide.

2. The composition of claim 1, wherein said polysaccharide is selected from the group consisting of alginate, chitosan, fucoidan, dextran, dextran sulfate, pentosan polysulfate, a carrageenan, a pectin, a pectin derivative, and a cellulose derivative and pectin derivatives, and cellulose derivatives.

3. The composition of claim 1, wherein said polysaccharide is alginate.

4. The composition of claim 1, wherein said cytokine-inhibiting molecule is selected from the group consisting of an antibody, an antibody fragment, a phage peptide, a receptor fragment, and a nucleic-based aptamer.

5. The composition of claim 1, wherein said cytokine-inhibiting molecule inhibits at least one of tumor necrosis factor alpha (TNF-α), interleukin-1a, interleukin-1b, interferon-g, interleukin-12, interleukin-23, transforming growth factor-b, interleukin-6, interleukin-2, and interleukin-4.

6. The composition of claim 3, wherein said alginate is selected from the group consisting of an unmodified alginate, an alkyl-substituted alginate, an aryl-substituted alginate, a propylene glycol-functionalized alginate, and a cross-linked alginate.

7. The composition of claim 3, wherein said cytokine-inhibiting molecule is anti-TNF-mAB and said alginate is an unmodified alginate.

8. A method for increasing the binding affinity of a cytokine-inhibiting molecule, comprising:
selecting a cytokine-neutralizing molecule having a binding affinity; and
covalently attaching a cytokine-inhibiting molecule to a polysaccharide, thereby increasing the binding affinity of said cytokine-neutralizing molecule.

9. The method of claim 8, wherein said polysaccharide is selected from the group consisting of alginate, chitosan, fucoidan, dextran, dextran sulfate, pentosan polysulfate, a carrageenan, a pectin, a pectin derivative, and a cellulose derivative and pectin derivatives, and cellulose derivatives.

10. The method of claim 8, wherein said polysaccharide is alginate.

11. The method of claim 8, wherein said cytokine-inhibiting molecule is selected from the group consisting of an antibody, an antibody fragment, a phage peptide, a receptor fragment, and a nucleic-based aptamer.

12. The method of claim 8, wherein said cytokine-inhibiting molecule inhibits at least one of tumor necrosis factor alpha (TNF-α), interleukin-1a, interleukin-1b, interferon-g, interleukin-12, interleukin-23, transforming growth factor-b, interleukin-6, interleukin-2, and interleukin-4.

13. The method of claim 10, wherein said alginate is selected from the group consisting of an unmodified alginate, an alkyl-substituted alginate, an aryl-substituted alginate, a propylene glycol-functionalized alginate, and a cross-linked alginate.

14. The method of claim 10, wherein said cytokine-inhibiting molecule is anti-TNF-α and said alginate is an unmodified alginate.

15. A method of treatment for a cytokine-related disorder, comprising:
administering to a patient in need of treatment a composition of claim 1.

16. A composition comprising:
a cytokine-inhibiting molecule; and
a synthetic polymer, wherein the cytokine-inhibiting antibody is covalently attached to the synthetic polymer, and wherein the synthetic polymer is selected from the group consisting of as poly(acrylic acid), poly(methacrylic acid), poly(acrylamide), a charged polystyrene derivative, a polyvinylpyrrolidone, a poly(amino acid), a poly(amines), and a polyelectrolyte.
